(19) United States
(12) Patent Application Publication
(10) Pub. No.: US 2014/0275816 A1
(43) Pub. Date: Sep. 18, 2014

(54) WIRELESS PATIENT MONITORING SYSTEM

(71) Applicant: COVIDIEN LP, Mansfield, MA (US)

(72) Inventor: Donald R. Sandmore, Lyons, CO (US)

(73) Assignee: COVIDIEN LP, Mansfield, MA (US)

(21) Appl. No.: 13/800,355

(22) Filed: Mar. 13, 2013

(51) Int. Cl. A61B 5/0205; A61B 5/01; A61B 5/00; A61B 5/0476; A61B 5/1455

(52) U.S. Cl. CPC A61B 5/02055; A61B 5/0476; A61B 5/14551; A61B 5/0006; A61B 5/01; A61B 5/6832
USPC 600/301; 600/300; 600/544; 600/323; 600/324; 600/476

(57) ABSTRACT

Embodiments of the present disclosure relate to medical systems having a plurality of separately wireless sensor elements. According to certain embodiments, the sensor elements may be physically separate from each other and may be configured to separately wirelessly communicate with a medical monitor. In some systems, two or more of the sensor elements may function together to monitor a physiological parameter. At least one of the sensor elements may include optical elements for pulse oximetry monitoring or regional saturation monitoring. In certain embodiments, at least one of the sensor elements may include electrodes for bispectral index monitoring.

### BACKGROUND

The present disclosure relates generally to medical devices and, more particularly, to wireless sensors for determining physiological parameters, such as plethysmographically-determined parameters and electroencephalography-derived parameters.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring certain physiological characteristics of a patient. Such devices provide doctors and other healthcare personnel with the information they need to provide the best possible healthcare for their patients. As a result, such monitoring devices have become an indispensable part of modern medicine. For example, photoplethysmography is a common technique for monitoring physiological characteristics of a patient, and one device based upon photoplethysmography techniques is commonly referred to as pulse oximetry. Pulse oximeters may be used to measure and monitor various blood flow characteristics of a patient. A pulse oximeter may be utilized to monitor the blood oxygen saturation of hemoglobin in arterial blood, the volume of individualized blood pulsations supplying the tissue, and/or the rate of blood pulsations corresponding to each heartbeat of a patient. In fact, the “pulse” in pulse oximetry refers to the time-varying amount of arterial blood in the tissue during each cardiac cycle.

A patient in a hospital setting may be monitored by a variety of medical devices, including devices based on pulse oximetry techniques. For example, a patient may be monitored with a pulse oximetry device, which may be appropriate for a wide variety of patients. Depending on the patient's clinical condition, a physician may also monitor a patient with a regional saturation monitor placed on the patient's head to determine if the patient is at risk of hypoxia. If a patient is scheduled for surgery, additional monitoring devices may be applied. One such device may include a sensor for bispectral index (BIS) monitoring to measure the level of consciousness by algorithmic analysis of a patient's electroencephalography (EEG) during general anesthesia. Examples of parameters assessed during the BIS monitoring may include the effects of anesthetics, evaluating asymmetric activity between the left and right hemispheres of the brain in order to detect cerebral ischemia, and detecting burst suppression. Such monitoring may be used to determine if the patient's anesthesia level is appropriate and to maintain a desired anesthesia depth.

Proper medical sensor placement may be difficult if multiple sensors (e.g., pulse oximetry, regional saturation sensors, and/or BIS monitoring sensors) are simultaneously used on the patient's tissue. Each type of sensor may include its own cable and, in some instances, its own dedicated monitor. Accordingly, the sensors, their cables, and/or their monitors may physically interfere with one another and may limit the ability to place multiple sensors on the patient's tissue. Additionally, the multiple components (e.g., emitters, detectors, electrodes, etc.) of each type of sensor are typically integrated into a single sensor body (e.g., BIS sensors have multiple electrodes integrated into a single sensor housing). Such configurations limit the range of options available for positioning the sensor components on the patient and limit the ability to replace or reposition the components of each sensor.

### BRIEF DESCRIPTION OF THE DRAWINGS

Advantages of the disclosed techniques may become apparent upon reading the following detailed description and upon reference to the drawings in which:

FIG. 1 is a perspective view of an embodiment of a monitoring system configured to be used with multiple wireless sensor elements;

FIG. 2 is a front view of a monitoring system configured to be used with wireless sensor elements having BIS sensor functionality in accordance with an embodiment;

FIG. 3 is a block diagram of a monitoring system configured to be used with multiple wireless sensor elements in accordance with an embodiment;

FIG. 4 is a front view of a plurality of wireless sensor elements in accordance with an embodiment coupled to a patient (e.g., six sensor elements having BIS, regional saturation, and pulse oximetry functionality);

FIG. 5 is a front view of a plurality of wireless sensor elements in accordance with an embodiment coupled to a patient (e.g., five sensor elements, including certain sensor elements coupled by a perforated edge);

FIG. 6. is a front view of a plurality of wireless sensor elements in accordance with an embodiment coupled to a patient (e.g., four sensor elements, including a sensor element having components for both regional saturation and pulse oximetry);

FIG. 7 is a front view of a plurality of wireless sensor elements in accordance with an embodiment coupled to a patient (e.g., four sensor elements, including two emitters for regional saturation techniques); and

FIG. 8 is a side cross-sectional view of a plurality of wireless sensor elements in accordance with an embodiment, taken along line 8-8 of FIG. 7.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The present disclosure is generally directed to monitoring systems for photoplethysmography and/or electroencephalography (EEG). The described multi-component systems may include an array of sensor elements (e.g., sensor components) that are physically separate (e.g., have a separate body) and are separately wireless, such that each sensor element is configured to be in separate wireless communication with an external device (e.g., a monitor). One or more of the plurality of sensor elements may be configured to alternatively or additionally communicate wirelessly with one or more other sensor elements in the monitoring system. The plurality of sensor elements may include optical elements configured to perform pulse oximetry and/or regional saturation measurements. The sensor elements may also include EEG electrodes for BIS monitoring and/or other components for collecting various types of physiological data (e.g., temperature, etc.). Thus, one of these sensor elements, or certain combinations of these sensor elements, may act to monitor one or more physiological parameters through pulse oximetry, regional saturation, and/or BIS monitoring. Additionally, at least one of the sensor elements may be configured to monitor more than one physiological parameter. Indeed, components (e.g., emitters, detectors, electrodes, etc.) for pulse oximetry, regional saturation, BIS monitoring, and/or other measurements may be arranged or combined in any suitable manner in any number of separately wireless sensor elements to facilitate patient monitoring.

Systems having the wireless sensor elements in accordance with the present disclosure may provide certain advantages over traditional wired sensors. For example, wireless sensor elements do not require cables, which reduces interference from such cables and also allows for increased mobility of a patient. Additionally, in some embodiments, the wireless sensor elements may also provide for separation of certain components that are typically included in a single sensor body (e.g., BIS sensors typically include four electrodes within a single housing or body), thus allowing more options for placing such components (e.g., electrodes) on the patient. Furthermore, in some embodiments, each of the wireless sensor elements may include components of multiple different types of sensors (e.g., one sensor element may include an electrode for BIS monitoring and an emitter for regional saturation measurements). Thus, components that are typically located in separate sensor bodies may be united into one sensor element structure. Such features may provide for increased flexibility and customization of the monitoring system, and may permit the system to be readily adapted for certain circumstances or for particular patients. Such features may further allow for easy removal or replacement of each sensor element.

With this in mind, FIG. 1 depicts an embodiment of a patient monitoring system 10 that includes a patient monitor 12 that may be used in conjunction with a sensor element 14 (e.g., one or more sensor elements 14 or a plurality of sensor elements 14). In particular, the monitor 12 may be used with an array 15 of sensor elements 14. The sensor elements 14 may be physically separate and individually wireless, such that each sensor element 14 may be configured to wirelessly communicate with external devices, such as the monitor 12. Although only four separate sensor elements 14a, 14b, 14c, 14d are shown in wireless communication with the monitor 12 in FIG. 1, in other embodiments, five, six, seven, eight, nine, ten, or more various sensor elements 14 may be in wireless communication with the monitor 12. Similarly, although one monitor 12 is depicted, two, three, four, or more similar or different monitors may be provided as part of the system 10.

In certain embodiments, one or more of the wireless sensor elements 14 may be completely or partially disposable. That is, in certain embodiments, a portion of the wireless sensor elements 14 may be disposed after patient use. In certain embodiments, the wireless sensor elements 14 may be constructed in a modular fashion such that portions of each sensor element 14 (e.g., an emitter portion, a detector portion, electrode portion, wireless transceiver portion, battery portion) may be removed to be recycled into other sensors while other portions of the sensor element 14 are disposed.

Additionally, each sensor element 14 may include a sensor body, which may function as a structural support for the components (e.g., emitters 16, detectors 18, electrodes 20, batteries, wireless transceivers, etc.). Each sensor element 14 may be formed from any suitable material or combination of materials, including rigid or conformable materials, such as fabric, paper, rubber, or elastomeric compositions. Furthermore, the sensor element 14 may include one or more layers (e.g., a base structural layer, an adhesive layer, and/or a foam layer). The various layers may include flexible polymeric materials (e.g., polyester, polyurethane, polypropylene, polyethylene, polyvinylchloride, acrylics, nitrile, PVC films, and acetates), foam materials (e.g., polyester foam, polyethylene foam, polyurethane foam, or the like), and adhesives (e.g., an acrylic-based adhesive, a supported transfer tape, an unsupported transfer tape, or any combination thereof). The sensor elements 14 may be self-adherent and self-prepping to facilitate applying the sensor elements 14 to the forehead and temple areas of the patient, for example.

As discussed herein, the various sensor elements 14 may be configured to monitor a physiological parameter. In particular embodiments, one or more of the sensor elements 14 may be configured to obtain photoplethysmography and/or pulse oximetry data. Thus, the sensor elements 14 may include various combinations of one or more optical components (such as one or more emitters 16 and/or one or more detectors 18). Additionally or alternatively, the system 10 may be configured to obtain a variety of other medical measurements with suitable components in the plurality of sensor elements 14. For example, one or more of the sensor elements 14 may be configured to for electroencephalography monitoring (e.g., bispectral index or BIS monitoring), and thus may include one or more electrodes 20 configured to obtain EEG data. One or more of the sensor elements 14 may also be configured to monitor various other physiological parameters, such as respiration rate, continuous non-invasive blood pressure (CNIBP), tissue water fraction, hematocrit, and/or water content. One or more of the sensor elements 14 may include additional functionality, such as temperature or pressure sensing functionality, for example.

Where the system 10 is configured for pulse oximetry monitoring, one or more of the sensor elements 14 may include one or more emitters 16 configured to transmit light. In addition, one or more sensor elements 14 may include one or more detectors 18 to detect light transmitted from the emitters 16 into a patient's tissue after the light has passed through the blood perfused tissue. The detectors 18 may generate a photoelectrical signal correlative to the amount of light detect. The emitter 16 and detector 18 configured for pulse oximetry monitoring may be disposed in a single sensor element 14 or may be disposed in different sensor elements 14, as described in more detail below. The emitter 16 may be a light emitting diode, a superluminescent light emitting diode, a laser diode or a vertical cavity surface emitting laser (VCSEL). Generally, the light passed through the tissue is selected to be of one or more wavelengths that are absorbed by the blood in an amount representative of the amount of the blood constituent present in the blood. The amount of light passed through the tissue varies in accordance with the changing amount of blood constituent and the related light absorption. For example, the light from the emitter 16 may be used to measure blood oxygen saturation, water fractions, hematocrit, or other physiological parameters of the patient. In certain embodiments, the emitter 16 may emit at least two (e.g., red and infrared) wavelengths of light. The red wavelength may be between about 600 nanometers (nm) and about 700 nm, and the IR wavelength may be between about 800 nm and about 1000 nm. However, any appropriate wavelength (e.g., green, yellow, etc.) and/or any number of wavelengths (e.g., three or more) may be used. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In addition, one or more sensor elements 14 may be configured for regional oximetry monitoring. Whereas pulse oximetry measures blood oxygen saturation based on changes in the volume of blood due to pulsing tissue (e.g., arteries), regional oximetry examines blood oxygen saturation within the venous, arterial, and capillary systems within a region of a patient. For example, a regional oximeter may include an emitter 16 and a detector 18 configured to be placed on a patient's forehead and may be used to calculate the oxygen saturation of a patient's blood within the venous, arterial, and capillary systems of a region underlying the patient's forehead (e.g., in the cerebral cortex). In certain embodiments, the interrogated region of patient tissue may include a particular location in the brain, the abdomen, the kidney, the liver, and/or any other suitable location. In regional saturation techniques, the emitter 16 may include at least two light emitting diodes (LEDs), each configured to emit at different wavelength of light, e.g., red or near infrared light. In one embodiment, the LEDs of the emitter 16 emit light in the range of about 600 nm to about 1000 nm. In a particular embodiment, one LED of the emitter 16 is configured to emit light at about 730 nm and the other LED of the emitter 16 is configured to emit light at about 810 nm.

In accordance with the present disclosure, the emitter 16 and the detector 18 configured for regional saturation monitoring may be disposed in one sensor element 14, or the emitter 16 and detector 18 may be disposed in separate sensor elements, as described in more detail below. The regional oximetry components of the system 10 may include one emitter 16 (which may have at least two LED's, each configured to emit a different wavelength of light) and two detectors 18, with one detector 18 relatively “close” (e.g., proximal) to the emitter 16 and one detector 18 relatively “far” (e.g., distal) from the emitter 16. Light intensity of multiple wavelengths may be received at both the “close” and the “far” detectors 18. For example, if two wavelengths are used, the two wavelengths may be contrasted at each location and the resulting signals may be contrasted to arrive at a regional saturation value that pertains to additional tissue through which the light received at the “far” detector passed (tissue in addition to the tissue through which the light received by the “close” detector passed, e.g., the brain tissue), when it was transmitted through a region of a patient (e.g., a patient's cranium). Surface data from the skin and skull may be subtracted out, to produce a regional oxygen saturation (rSO₂) value for deeper tissues.

It is also contemplated that one or more sensor elements 14 may be configured for BIS monitoring. BIS is a measure of a patient's level of consciousness during general anesthesia, and BIS sensors are often applied to a patient's forehead during surgical procedures. BIS sensors may include multiple electrodes 20 to obtain electroencephalography (EEG) data. BIS monitoring may involve placing four or more electrodes 20 (e.g., ground electrode, artifact-measuring electrode, etc.) on the patient's tissue, such as on the patient's forehead. The electrodes 20 may be formed from a suitable conductive composition, such as a metal or alloy (e.g., silver/silver chloride, copper, aluminum, gold, or brass) or a conductive polymer. In the present embodiments, one or more electrodes 20 configured for BIS monitoring may be disposed in one sensor element 14, or the electrodes 20 may be disposed in two or more separate sensor elements 14, as discussed in detail below. Techniques for BIS monitoring may be as provided in U.S. Provisional Application No. 61/301,088, filed Feb. 3, 2010, and U.S. patent application Ser. No. 13/020,704, “Combined Physiological Sensor Systems and Methods,” which are hereby incorporated by reference herein in their entirety for all purposes.

With the foregoing in mind, the monitoring system 10 depicted in FIG. 1 includes the array 15 of wireless sensor elements 14 that together are configured for pulse oximetry, regional saturation, and BIS monitoring. Thus, the sensor elements 14 include various components for such monitoring, including emitters 16, detectors 18, and/or electrodes 20. In the particular embodiment of FIG. 1, a sensor element 14a (e.g., a central sensor element) may include one or more emitters 16 and one or more electrodes 20. The emitter 16a of the sensor element 14a may be configured to emit light for regional saturation monitoring, for example. The central electrode 20a of the sensor element 14a may be configured for BIS monitoring. Thus, the sensor element 14a may be configured for monitoring at least two physiological parameters (e.g., regional saturation and BIS monitoring). The sensor element 14a may be configured to be positioned generally high and central on the patient's forehead, as illustrated in FIG. 4, for example.

The system 10 of FIG. 1 may also have a sensor element 14b that includes electrodes 20 configured for BIS monitoring. In particular, the sensor element 14b may include a temple electrode 20b and an artifact-measuring electrode 20c configured to measure artifacts resulting from muscular movements (e.g., eye twitching). Although the temple electrode 20b and artifact-measuring electrode 20c are coupled together in the second sensor element 14b in the illustrated embodiment, these electrodes 20b, 20c may alternatively be disposed in separate sensor elements 14. Additionally, a ground electrode 20d may be disposed in a separate sensor element 14, or may be coupled to the sensor element 14a (as shown) or the sensor element 14b, or may be positioned in any suitable location. Thus, in this embodiment, the sensor element 14a and the sensor element 14b may function together for BIS monitoring.

The system 10 depicted in FIG. 1 may also include a sensor element 14c. The sensor element 14c may include a first detector 18a and a second detector 18b configured to detect light reflected from the patient's tissue. In particular, the two detectors 18a, 18b of the sensor element 14c are configured to detect light emitted by emitter 16a of the sensor element 14a in order to obtain regional saturation data. Thus, in this embodiment, the central sensor element 14a and the sensor element 14c may function together for regional saturation monitoring. As described in more detail below, regional saturation monitoring may require certain distances between the emitter 16a and the detectors 18a, 18b. In some embodiments, the sensor elements 14a, 14c may be in wireless communication with each other, and the system 10 may be configured to determine whether the sensor elements 14a, 14c (or the optical components, such as the emitter 16 and the detector 18, disposed therein) are properly spaced and positioned at suitable relative locations on the patient for regional saturation monitoring. As shown, the system 10 may also include a sensor element 14d that is configured for pulse oximetry monitoring. Thus, the sensor element 14d of the illustrated embodiment includes an emitter 16b and a detector 18c configured to measure the blood oxygen saturation of the patient. Additionally, any suitable configuration and combination of emitters 16, detectors 18, and/or electrodes 20 disposed within various wireless sensor elements 14 is envisioned. Several different embodiments of the system 10 having sensor elements 14 are described in more detail below.

Regardless of the configuration of the sensor elements 14, each of the sensor elements 14 may be configured to separately wirelessly communicate 22 with one or more external devices. In other words, each sensor element 14 may include, or may be coupled to, a wireless transceiver that facilitates wireless communication 22 with the monitor 12, as shown in FIG. 1. The monitor 12 may be any suitable monitor, such as a pulse oximetry monitor available from Covidien LP. The monitor 12 may include a monitor display 24 configured to display information regarding the physiological parameters, information about the system, and/or alarm indications, for example. The monitor 12 may also include various input components 26, such as knobs, switches, keys and keypads, buttons, etc., to provide for operation and configuration of the monitor 12 and monitoring system 10. The monitor 12 may include a wireless module 28 for transmitting and receiving wireless data, a memory, a processor, and various monitoring and control features.

In certain embodiments, the physiological parameter of the patient may be calculated by the wireless sensor elements 14. However, as discussed in detail below, in certain embodiments the patient monitor 12 may calculate the physiological parameter instead of, or in addition to, the sensor elements 14. The monitor 12 may also be coupled to a multi-parameter monitor 30 via a cable 32 connected to a sensor input port or via a cable 34 connected to a digital communication port. In addition to the monitor 12, or alternatively, the multi-parameter monitor 22 may be configured to calculate physiological parameters and to provide a central display 36 for visualization of information from the monitor 12 and from other monitoring devices or systems. The multi-parameter monitor 30 may facilitate presentation of patient data, such as pulse oximetry data determined by system 10 and/or physiological parameters determined by other patient monitoring systems (e.g., electrocardiographic (ECG) monitoring system, a respiration monitoring system, a blood pressure monitoring system, etc.). For example, the multi-parameter monitor 30 may display a graph of SpO₂ values, a current pulse rate, a graph of blood pressure readings, an electrocardiograph, and/or other related patient data in a centralized location for quick reference by a medical professional. Although cables 32 and 34 are illustrated, it should be understood that the monitor 12 may be in wireless communication with the multi-parameter monitor 30.

The wireless transceiver/receivers of the sensor elements 14 and the wireless module 28 of the monitor 12 may be configured to communicate using the IEEE 802.15.4 standard, and may be, for example, ZigBee, WirelessHART, or MiWi modules. Additionally or alternatively, the wireless module 28 may be configured to communicate using the Bluetooth standard, one or more of the IEEE 802.11 standards, an ultra-wideband (UWB) standard, or a near-field communication (NFC) standard. As described further below, the sensor elements 14 may wirelessly transmit either raw detector signals or calculated physiological parameter values to the patient monitor 12. Additionally, the monitor 12 may use the wireless module 28 to send the sensor elements 14 instructions and/or operational parameters set by the operator using the monitor 12.

As previously indicated, certain embodiments of the system 10 may include one or more sensor elements 14 that are configured for BIS monitoring. Indeed, in some embodiments, a wireless BIS sensor having one or more electrodes 20 may be provided. In some embodiments, the one or more electrodes 20 may be disposed in physically separate wireless sensor elements 14. In systems 10 having BIS functionality, an EEG monitor 38 (e.g., a BIS monitor) may be provided, and sensor elements 14 having BIS sensor components (e.g., electrodes 20 and associated circuitry) may wirelessly communicate with the BIS monitor 38. One embodiment of the BIS monitor 38 is illustrated in FIG. 2. In the particular embodiment depicted, the BIS monitor 38 is in wireless communication 40 with a sensor element 14a having a central electrode 20a, and a sensor element 14b having the temple electrode 20b and artifact-measuring electrode 20c disposed therein. The BIS monitor 38 may be provided in lieu of, or in addition to, the patient monitor 12. In certain embodiments, the BIS monitor 38 may be adapted to receive, process, and display other patient parameters (e.g., pulse oximetry, regional saturation, blood pressure, temperature, etc.). However, in some embodiments, the patient monitor 12 may be adapted to receive, process, and display BIS measurements. In other words, the BIS-related displays and functionality of the BIS monitor 38 may be integrated into the monitor 12. Thus, various configurations and combinations of the monitor 12 and BIS monitor 38 are envisioned for use in the presently described systems 10.

In general, the BIS monitor 38 may be configured to calculate physiological characteristics relating to the EEG signal received from the BIS sensor components (e.g., one or more electrodes 20). For example, the BIS monitor 38 may be configured to algorithmically calculate BIS from the EEG signal. As noted above, BIS is a measure of a patient's level of consciousness during general anesthesia. Further, the BIS monitor 38 may include a display 42 configured to display physiological characteristics, historical trends of physiological characteristics, other information about the system (e.g., instructions for placement of the BIS electrodes 20 on the patient), and/or alarm indications. For example, the BIS monitor 38 may display a patient's BIS value 44. The BIS value 44 represents a dimensionless number (e.g., ranging from 0, i.e., silence, to 100, i.e., fully awake and alert) output from a multivariate discriminate analysis that quantifies the overall bispectral properties (e.g., frequency, power, and phase) of the EEG signal. For example, a BIS value 44 between 40 and 60 may indicate an appropriate level for general anesthesia. The BIS monitor 38 may also display a signal quality index (SQI) bar graph 46 (e.g., ranging from 0 to 100) which measures the signal quality of the EEG channel source(s) based on impedance data, artifacts, and other variables. The BIS monitor 38 may also display an electromyograph (EMG) bar graph 48 (e.g., ranging from 30 to 55 decibels) which indicates the power (e.g., in decibels) in the frequency range of 70 to 110 Hz. The frequency range may include power from muscle activity and other high-frequency artifacts. The BIS monitor 38 may further display a suppression ratio (SR) 50 (e.g., ranging from 0 to 100 percent), which represents the percentage of epochs over a given time period (e.g., the past 63 seconds) in which the EEG signal is considered suppressed (i.e., low activity). In certain embodiments, the BIS monitor 38 may also display a burst count for the number of EEG bursts per minute, where a “burst” is defined as a short period of EEG activity preceded and followed by periods of inactivity or suppression. The BIS monitor 38 may also display the EEG waveform 52. In certain embodiments, the EEG waveform 52 may be filtered. The BIS monitor 38 may also display trends 54 over a certain time period (e.g., one hour) for EEG, SR, EMG, SQI, and/or other parameters. In certain embodiments, the BIS monitor 38 may store instructions on a memory specific to a specific sensor element 14 or electrode 20 type or model.

Additionally, the BIS monitor 38 may include various activation mechanisms 56 (e.g., buttons and switches) to facilitate management and operation of the BIS monitor 38. For example, the BIS monitor 38 may include function keys (e.g., keys with varying functions), a power switch, adjustment buttons, an alarm silence button, and so forth. It should be noted that in other embodiments, the parameters described above and the activation mechanisms 56 may be arranged on different parts of the BIS monitor 38. In other words, the parameters and activation mechanisms 56 need not be located on a front panel 58 of the BIS monitor 38. Indeed, in some embodiments, activation mechanisms 56 are virtual representations in a display or actual components disposed on separate devices. In addition, the activation mechanisms 56 may allow selecting or inputting of a specific sensor type or model in order to access instructions stored within the memory of the sensor element 14.

Separately wireless sensor elements may communicate with the monitor 12 as shown in FIG. 3. In particular, FIG. 3 depicts a block diagram of one embodiment of a patient monitoring system 10 having a plurality of sensor elements 68 configured for both pulse oximetry and regional saturation monitoring of a patient 70. As shown, each of the sensor elements 68 may be in separate wireless communication with the monitor 12. Although three sensor elements 68a, 68b, 68c, are depicted, four, five, six, or more sensor elements 68 may be included in the system 10, and any of the sensor elements 68 may be configured to have BIS functionality. Similarly, although one monitor 12 is depicted, two, three, four, or more similar or different monitors (e.g., BIS monitor 38) may be provided as part of the system 10.

In the particular embodiment of FIG. 3, a sensor element 68a may have one or more emitters 16b and one or more detectors 18c configured for pulse oximetry monitoring. A sensor element 68b may have a plurality of detectors 18a, 18b, and a sensor element 68c may have an emitter 16a, wherein the second and third sensor elements 68b, 68c are configured to function together for regional saturation monitoring.

Regardless of the particular sensing components included in the various sensor elements 68, each sensor element 68 may include or may be coupled to a battery 72 to supply the sensor element 14 with power. By way of example, the battery 72 may be a rechargeable battery (e.g., a lithium ion, lithium polymer, nickel-metal hydride, or nickel-cadmium battery) or may be a single-use battery such as an alkaline or lithium battery. Since a battery 72 may be required for each wireless sensor element 68, the battery 72 may be much smaller, and accordingly may have a lower capacity and be less expensive, than a battery needed to power a larger wireless sensor (e.g., a wireless sensor have multiple optical components or multiple electrodes of a BIS sensor) that does not employ the disclosed techniques. A battery meter may be included in some or all of the sensor elements 68 to provide the expected remaining power of the battery 72 to the monitor 12.

Each sensor element 68 may also include an encoder 74 that may provide signals indicative of the wavelength of one or more light sources of the emitters 16, which may allow for selection of appropriate calibration coefficients for calculating a physical parameter such as blood oxygen saturation. The encoder 74 may, for instance, be a coded resistor, EEPROM or other coding devices (such as a capacitor, inductor, PROM, RFID, parallel resident currents, or a colorimetric indicator) that may provide a signal to a microprocessor 76 of the monitor 12 related to the characteristics of the sensor element 68 to enable the microprocessor 76 to determine the appropriate calibration characteristics. In some embodiments, the encoder 74 and/or the decoder 78 may not be present.

Additionally, each sensor element 14 may include or may be coupled to a wireless transceiver 80 to send data to the monitor 12 or to receive instructions from the monitor 12. The monitor 12 may also include a wireless transceiver 66. In general, when data is sent from the sensor element 68 and received by the monitor 12, the patient monitor 12 may determine which type of data has been received. For example, the monitor may determine whether the data is pulse oximetry data or regional saturation data. As such, data received from the sensor element 68 may be stored in RAM 82 so that the microprocessor 76 may examine the received data to determine whether it is pulse oximetry data, regional saturation data, or another type of data (e.g., EEG data, temperature data, etc.).

Signals from the detector 18 and/or the encoder 74 may be wirelessly transmitted to the monitor 12. The monitor 12 may include one or more microprocessors 76 coupled to an internal bus 84. Also connected to the bus may be a ROM memory 86, a RAM memory 82 and a display 24. A time processing unit (TPU) 88 may provide timing control signals to light drive circuitry 90, which controls when the emitter 16 is activated, and if multiple light sources are used, the multiplexed timing for the different light sources. It is envisioned that the emitters 16 may be controlled via time division multiplexing of the light sources. TPU 88 may also control the gating-in of signals from detector 18 through a switching circuit 92. These signals are sampled at the proper time, depending at least in part upon which of multiple light sources is activated, if multiple light sources are used. The received signal from the detector 18 may be passed through an amplifier 94, a low pass filter 96, and an analog-to-digital converter 98 for amplifying, filtering, and digitizing the electrical signals received from sensor element 14. The digital data may then be stored in a queued serial module (QSM) 100, for later downloading to RAM 82 as QSM 100 fills up. In an embodiment, there may be multiple parallel paths for separate amplifiers, filters, and A/D converters for multiple light wavelengths or spectra received.

In one embodiment, based at least in part upon the received signals corresponding to the light received by the detectors 18, the microprocessor 76 may calculate the oxygen saturation and regional oxygen saturation using various algorithms. These algorithms may use coefficients, which may be empirically determined. For example, algorithms relating to the distance between the emitter 16 and various detector elements in the detector 18 may be stored in a ROM 86 and accessed and operated according to microprocessor 76 instructions.

Furthermore, one or more functions of the monitor 12 may also be implemented directly in one or more of the sensor elements 68. For example, in some embodiments, one or more of the sensor elements 68 may include one or more processing components configured to calculate the physiological characteristics from the signals obtained from the patient. One or more of the sensor elements 68 may have varying levels of processing power, and may wirelessly output data in various stages to the monitor 12. For example, in some embodiments, the data output to the monitor 12 may be analog signals, such as detected light signals (e.g., pulse oximetry signals or regional saturation signals), or processed data.

With the foregoing in mind, FIGS. 4-8 illustrate various embodiments of patient monitoring systems having arrays of sensor elements. In addition to the specific components depicted, each of the sensor elements may include any suitable additional components, such as a battery 72 (e.g., a rechargeable battery), a battery meter, an encoder 74, and/or a wireless transceiver/receiver 80 so as to independently communicate wirelessly with one or more associated patient monitors (e.g., the patient monitor 12 and/or the BIS monitor 38). It should be understood that each system 10 may include additional or few sensor elements, and that each sensor element may include any additional suitable components for patient monitoring, such as emitters 16, detectors 18, BIS electrodes 20, and the like.

FIG. 4 depicts one embodiment of a patient monitoring system 120 including an array 121 of separate wireless sensor elements 122. As shown, the system 120 includes six separate sensor elements 122a, 122b, 122c, 122d, 122e, 122f, each containing various components (e.g., emitters 16, detectors 18, electrodes 20) for monitoring physiological parameters of a patient. In particular, a sensor element 122a (e.g., a central sensor element) may include a sensor element body 124 configured to be applied to the patient's forehead. The sensor element 122a may include one or more central electrodes 20a for BIS monitoring. In some embodiments, the sensor element 122a may additionally include one or more central emitters 16a. In one embodiment, the central emitter 16a may be configured for use in regional saturation monitoring. To that end, the central emitter 16a may include at least two light emitting diodes (LEDs), each configured to emit a different wavelength of light, e.g., red or near infrared light. The sensor element 122a may be designed for placement along a central axis 126 of the patient's forehead, and both the central electrode 20a and central emitter 16a may be arranged/aligned within the sensor element 122a so as to substantially align with the central axis 126 of the patient's forehead when the sensor element 122a is applied to the patient. A grounding portion 128, which includes a grounding electrode 20d, may be coupled to the sensor element 122a, as shown in FIG. 4. However, it should be understood that the grounding electrode 20d may be coupled to any of the sensor elements 122 of the system 120.

Furthermore, the system 120 may include a sensor element 122b that includes one or more detectors 18 configured to detect light at various intensities and wavelengths. As shown, two detectors 18a, 18b may be configured to detect light emitted from the central emitter 16a of the sensor element 122a after the light passes through the tissue of the patient. After converting the received light into an electrical signal, the detectors 18a, 18b may wirelessly send the signals to the monitor 12, where physiological characteristics (e.g., regional saturation) may be calculated based at least in part on the absorption and/or reflection of light by the tissue of the patient. Thus, in certain embodiments, the central emitter 16a of the sensor element 122a and the detectors 18a, 18b of the sensor element 122b may function together to for regional saturation monitoring. The sensor element 122b may be configured so that when applied to the patient, the one or more detectors 18a, 18b are disposed along a horizontal axis 130 of the patient's forehead and are aligned with the central emitter 16a of the central sensor element 122a. Furthermore, the sensor element 122b may be configured such that when applied to the patient, the first detector 18a is a first distance, D₁, from the central emitter 16a. Additionally, the second detector 18b may be a second distance D₂ from the central emitter 16a, wherein the distance D₂ is shorter than the distance D₁. For regional saturation measurements, the distance D₁ represents a shallower optical path and the distance D₂ represents a deeper optical path for cranial penetration. In certain embodiments, when applied to the patient, the first distance D₁ is about 75% of the second distance D₂. In a particular embodiment, the first distance D₁ is about 30 mm while the second distance D₂ is about 40 mm. In other embodiments, the first distance D₁ may be between about 1 to about 3 cm, while the second distance D₂ may be about 3 to about 4 cm. Thus, the detectors 18a, 18b on the sensor element 122b may be spaced about 10 mm apart. In general, the sensor element 122b may be configured so that suitable distances between the emitters 16 and the detectors 18 can be achieved.

As noted above, in some embodiments, one or more of the sensor elements 122 may be in wireless communication with one another. Such a configuration may enable the system 10 to have fewer than all of the sensor elements 122 in direct communication with the monitor 12. For example, a first sensor element 122 disposed on the patient may provide information (e.g., physiological data, physiological signals, physiological parameters, position information, relative position or distance information, calibration information, etc.) to a second sensor element 122 (e.g., a second sensor element 122 that is configured to receive information from one or more other sensor elements 122) disposed on the patient, and the second sensor element 122 in turn relays the provided information to the monitor 12. Such wireless communication between the sensor elements 122 may also enable the system 10 to determine various characteristics of the sensor elements 122, such as whether the sensor elements 122 are properly spaced and/or positioned at suitable relative locations on the patient. For example, the first sensor element 122 may be configured to wirelessly communicate with the second sensor element 122, and the first and/or the second sensor element 122 may be configured to determine (or provide information that enables the monitor 12 to determine) whether the sensor elements 122 (or the components therein) are properly spaced with respect to one another and/or at suitable locations relative to one another and/or the patient. For example, the first and/or the second sensor element 122 may be configured to process certain received information to determine whether the first and the second sensor elements 122 are properly spaced with respect to one another and/or at suitable locations relative to each other and/or the patient. In some embodiments, the first and/or the second sensor element 122 may also communicate with the monitor 12. For example, the first sensor element 122 may be configured to relay information from the second sensor element 122, to provide information related to the spacing and/or location of the sensor elements 122, and/or to indicate to the monitor 12 that the first and second sensor elements 122 are properly spaced and/or located on the patient.

In certain embodiments, one sensor element 122 may receive information from a plurality of communicatively-coupled sensor elements 122 on the patient and relay the received information to the monitor 12. More particularly, multiple sensor elements 122 (such as the sensor elements 122b, 122c, 122d, 122e, and/or 122f, for example) may be configured to wirelessly communicate with another sensor element 122 (such as the central sensor element 122a), which in turn may be configured to receive and to relay the information collected by the communicatively-coupled sensor elements 122 (such as 122a, 122b, 122c, 122d, 122e, and/or 122f) to the monitor 12.

In some embodiments, one or more of the sensor elements 122a, 122d, 122e that are configured for BIS monitoring may be configured to wirelessly communicate with one another. In some embodiments, the sensor elements 122d, 122e may provide information to the central sensor element 122a, which in turn relays the information to the monitor 12. In certain embodiments, one or more of the sensor elements 122a, 122b, 122c that are configured for regional saturation monitoring may be configured to wirelessly communicate with one another. For example, in some embodiments, the sensor elements 122b, 122c may be configured to communicate information to the central sensor element 122a, which in turn relays the information to the monitor 12. In some embodiments, one or more of the sensor elements 122b, 122c, 122d, 122e, 122f may wirelessly communicate information to the central sensor element 122a, which may in turn relay the information to the monitor 12. In yet another embodiment, one or more of the sensor elements 122a, 122b, 122c, 122d, 122e may wirelessly communicate information to the sensor element 122f that is configured for pulse oximetry monitoring, which may in turn relay the information to the monitor 12. Although specific examples of suitable configurations of communicatively-coupled sensor elements 122 are provided herein, any configuration that enables communication between sensor elements 122 is envisioned. Additionally, in some circumstances, the system 10 may be configured to begin the monitoring session (e.g., collect physiological data) only if the system 10 has positively determined that the sensor elements 122 are at the proper relative spacing and/or locations, or in some embodiments, the monitor 10 may be configured to provide an indication (e.g., a visual or audible signal, alarm, or alert) that the sensor elements 122 are not properly spaced and/or located, for example.

Additionally, in some embodiments, a sensor element 122c may be provided. Like the sensor element 122b, the sensor element 122c may include two detectors 18a, 18b configured to detect light at various intensities and wavelengths. The detectors 18a, 18b may detect light emitted by the central emitter 16a of the sensor element 122a after the light passes through the tissue of the patient. After converting the received light into an electrical signal, the detectors 18a, 18b may wirelessly send the signals to the monitor 12, where physiological characteristics may be calculated. Additionally, the sensor element 122c may be configured so that when applied to the patient the detectors 18a, 18b are disposed along the horizontal axis 130 of the patient's forehead and in line with the central emitter 16a of the sensor element 122a, and the detectors 18a, 18b may be at suitable distances (e.g., D₁ and D₂, respectively) from the central emitter 16a as described above with respect to the sensor element 122b.

In certain embodiments, as shown in FIG. 4, the sensor element 122c may be structurally and/or functionally similar to the sensor element 122b, although the sensor element 122c is configured to be disposed in a different location on the patient's forehead. For example, when applied to the patient, the sensor element 122b may be disposed on one side of the sensor element 122a (i.e., on a first side of the longitudinal axis 126), while the sensor element 122c may be disposed on the other side of the sensor element 122a (i.e., on a second side of the longitudinal axis 126). The use of the sensor elements 122b, 122c in this configuration may allow for dual or bilateral examination and may provide useful comparative regional saturation information. The output from each of the sensor elements 122b, 122c may be separately processed to provide a particular regional oxygen saturation value. These regional values may be separately displayed on the monitor display 24 as both a numeric or other such quantified value, for example, constituting basically an instantaneous real-time value, and as a point in a graphical plot, representing a succession of such values taken over time. While the instantaneous quantified value provides useful information, the graphical trace displays also provide useful information by directly showing an ongoing trend, and doing so in a contrasting, comparative manner for the multiple sensor elements 122b, 122c having detectors 18 for obtaining regional saturation data.

While the sensor elements 122b, 122c of FIG. 4 are structurally and functionally similar as depicted, the sensor elements 122b, 122c may also be structurally and/or functionally different from one another. For example, one of the sensor elements 122b, 122c may include an additional emitter 16 or detector 18, or one of the sensor elements 122b, 122c may include different components such as electrodes, temperature sensors, pulse oximetry sensor components, or the like. Furthermore, the sensor elements 122b, 122c may include different size and/or shapes, which may be helpful for accommodating the plurality of various sensor elements 122 on the patient.

In the embodiment of FIG. 4, the system 120 also includes a sensor element 122d configured to be disposed on or near one of the patient's temples, as shown. The sensor element 122d may include a temple electrode 20b. The sensor element 122d may further include an artifact-detecting electrode 20c configured to monitor artifacts resulting from muscular movement, such as eye twitching. In such embodiments, the temple electrode 20b and the artifact-detecting electrode 20c may be coupled together through any suitable means, including a cable or flex circuit 132, for example. Although these electrodes 20b, 20c may be disposed in physically separate wireless sensor elements 122, such a configuration shown in FIG. 4 may allow the temple and artifact-detecting electrodes 20b, 20c to easily share a battery and a wireless transceiver designated for the sensor element 122d, rather than each of these electrodes 20b, 20c being disposed in their own sensor element 122 and having their own battery and wireless transceiver, for example. Sharing these components may beneficially reduce the number of batteries and wireless transceivers/receivers (and thus, also reduce the size, weight, and cost) required for operation of the system 120.

The system 120 depicted in FIG. 4 may further include a sensor element 122e. As shown, the sensor element 122e may include a temple electrode 20b and an artifact-detecting electrode 20c configured to monitor artifacts resulting from muscular movement, such as eye twitching. The sensor element 122e may be configured to be disposed on or near the patient's other temple, thus creating a system 120 with bilateral BIS functionality. As in the sensor element 122e, the temple electrode 20b and the artifact-detecting electrode 20c may be coupled together through any suitable means, including a cable or flex circuit 132, for example. Again, such a configuration may allow the components (e.g., electrodes 20b, 20c) to share a battery and/or a wireless transceiver if desired, although these components may be provided separate batteries and/or wireless transceivers. While the sensor element 122e and the sensor element 122e of FIG. 4 are structurally and functionally similar, it is envisioned that these sensor elements 122d, 122e may also be structurally and/or functionally different from each other. For example, one of the sensor elements 122d, 122e may include different components such as additional electrodes, temperature sensors, emitters, detectors, or the like. Furthermore, one of the sensor elements 122d, 122e may have a different size and/or shape.

The system 120 of FIG. 4 may also include a sensor element 122f configured for pulse oximetry monitoring. The sensor element 122f may therefore take any form suitable for pulse oximetry. For example, as illustrated, the sensor element 122f may include one or more emitters 16b and one or more detectors 18c (not shown in FIG. 4) and may have a clip-style structure configured to be coupled to the patient's ear. The output from the sensor element 122f may be separately processed to provide various physiological parameters, such as oxygen saturation values, for example.

In accordance with the present disclosure, one or more additional pulse oximetry sensors (or suitable optical components within one or more sensor elements 122) may be employed to obtain oxygen saturation data from different points on the patient's body, such as a finger or toe, for example. The additional sensor or sensor element 122 may also be clip-style or wrap style sensor and may operate in reflectance, or transmittance mode, for example. Furthermore, where multiple pulse oximetry sensors are positioned on the patient's body (e.g., at different distances from the patient's heart), continuous non-invasive blood pressure (CNIBP) measurements may be calculated. Thus, the system 120 (or any of the systems described herein) may be configured to obtain pulse oximetry data from two different locations on the patient so that CNIBP may be determined. The various pulse oximetry sensors utilized for CNIBP may be configured to independently communicate wirelessly with one or more associated patient monitors (e.g., the patient monitor 12 and/or the BIS monitor 38).

FIG. 5 depicts an alternate embodiment of a monitoring system 150 having an array 151 of physically separate wireless sensor elements 152 for obtaining various physiological parameters. In the illustrated embodiment, a sensor element 152a may be similar to the sensor element 122a depicted in FIG. 4 and may include a central emitter 16a and a central electrode 20a. However, as shown, the sensor element 152a may be removably coupled to a sensor element 152b and/or a sensor element 152c. The sensor elements 152b, 152c may each include two or more detectors 18a, 18b configured for regional saturation monitoring. The sensor element may 152a be configured to be aligned along the central longitudinal axis 126 of the patient's forehead (e.g., both the central electrode 20a and the central emitter 16a are aligned along a central axis of the central sensor element 152a, which may preferably coincide with the central longitudinal axis 126 of the patient's forehead when the central sensor element 152a is applied to the patient as shown in FIG. 5). Moreover, one or both of the sensor elements 152b, 152c may be coupled to the sensor element 152a such that the central emitter 16a of the central sensor element 152a and each of the detectors 18a, 18b of the sensor elements 152b, 152c are aligned along the horizontal axis 130. Such relative positions of the sensor elements 152 may facilitate the collection of regional saturation data in the embodiment depicted.

The sensor element 152a and one or both of the sensor elements 152b, 152c may be removably coupled, such as by a perforated edge 154, for example. Thus, one or both of the sensor elements 152b, 152c may be easily separated from the sensor element 152a. In some cases, one or both of the sensor elements 152b, 152c may be separated from the sensor element 152a prior to placing the sensor elements 152 on the patient. However, in some cases, the sensor elements 152 may be placed on the patient as a single unit of attached sensor elements 152, and the various portions or sensor elements 152 may be removed for replacement, repair, or to adapt the system 150 to the particular monitoring needs of the patient. For example, all three sensor elements (i.e., sensor elements 152a, 152b, and 152c) may be placed on the patient at the beginning of a monitoring session as a single unit. However, it may be determined that the sensor element 152b is no longer functioning or is no longer needed. In that case, the sensor element 152b may be detached from the sensor element 152a and removed from the patient. If needed, a replacement sensor element 152b, or a different type of sensor element 152 (i.e., a sensor element having different functionality and/or different components such as pulse oximetry components or temperature sensors, for example) may be substituted for the removed sensor element 152b. Thus, the system 150 and the various sensor elements 152 therein may be changed and adapted as needed.

In the embodiment of FIG. 5, the sensor elements 152a, 152b, 152c may each have a separate battery and a wireless transceiver so that each sensor element 152 is separately powered and separately wireless. However, in an alternate embodiment, the removably coupled sensor elements 152a, 152b, 152c may share a single battery and/or a wireless transceiver (e.g., each sensor element 152 is coupled to the same battery and/or wireless transceiver). As explained above, sharing such components between one or more sensor elements 152 may reduce the size and cost of the system 150, and may make the system 150 smaller and more comfortable for the patient. In such cases, when one of the sensor elements 152 is replaced, a newly added sensor element 152 may be coupled to the shared battery and/or shared wireless transceiver. For example, the replacement sensor element 152 may be plugged in or electrically connected to the shared battery and/or shared wireless transceiver.

Furthermore, the embodiment of FIG. 5 includes a sensor element 152d, which may have a temple electrode 20b and an artifact-detecting electrode 20c. The sensor element 152d may be configured to be disposed on or near one of the patient's temples. In certain embodiments, the temple electrode 20b and the artifact-detecting electrode 20c may be coupled together through any suitable means, including a cable or flex circuit 132, for example. As explained above with respect to FIG. 4, such a configuration allows the temple and artifact-detecting electrodes 20b, 20c to share a battery and a wireless transceiver for the fourth sensor element 152d, rather than each of these electrodes 20b, 20c having their own battery and wireless transceiver, for example. Such configurations may beneficially reduce the number of batteries and wireless transceivers/receivers (and thus, also reduce the size, weight, and cost) required for operation of the system 150.

The embodiment of FIG. 5 may also have a sensor element 152e configured for pulse oximetry monitoring. As discussed above, such sensor elements 152 may take any suitable form for obtaining pulse oximetry data. For example, in the depicted embodiment, the sensor element 152e includes an emitter 16b and a detector 18c, and the sensor element 152e may be secured to the patient's forehead using any suitable attachment means, such as a headband 160. In such cases, the headband 160 may attach to the sensor element 152e, or instead, the headband 160 may be wrapped or disposed over the sensor element 152e, applying pressure over the pulse oximetry components (e.g., emitter 16b and detector 18c) of the sensor element 152e to ensure that the emitter 16b and detector 18c are adequately coupled to the patient's forehead.

FIG. 6 depicts yet another embodiment of a monitoring system 170 in accordance with the present disclosure. As illustrated, the system 170 includes an array 171 of physically separate wireless sensor elements 172. A sensor element 172a (e.g., a central sensor element) may be similar to the sensor elements 122a and 152a discussed above. In particular, the sensor element 172a may include a central electrode 20a and a central emitter 16a. A sensor element 172b and/or a sensor element 172c may be provided, each having at least two detectors 18a, 18b for regional saturation monitoring as discussed above with respect to FIGS. 1-5. In the embodiment of FIG. 6, the sensor element 172b may further include an emitter 16b such that pulse oximetry monitoring may be carried out using the emitter 16b and one or both detectors 18a, 18b of the sensor element 172b. In such embodiments, the emitter 16b may be configured such that, when applied to the patient, the emitter 16b is relatively closer to the patient's lower forehead region in order to take advantage of the relatively better blood perfusion characteristics in that region. Thus, pulse oximetry measurements may be obtained without an additional separate sensor element 172 for this purpose (e.g., without the sensor element 122f of FIG. 4 or the sensor element 152e of FIG. 5). Such a configuration may result in a system 170 that is smaller and more comfortable for the patient, and the system may be lower cost as at least some of the components (e.g., emitters 16 and detectors 18) for regional saturation and pulse oximetry measurements are shared. Furthermore, combining components (e.g., emitters 16 and detectors 18) in such a manner within one sensor element 172 may reduce the number of batteries and wireless transceivers required for the system 170. It should be understood that the emitter 16b for pulse oximetry monitoring may additionally or alternatively be disposed in the sensor element 172c. The system 170 of FIG. 6 may also include a sensor element 172d having a temple electrode 20b and/or an artifact-detecting electrode 20c, as shown. The sensor element 172d may be similar to the sensor elements 122d, 122e of FIG. 4, for example.

The emitter 16a utilized for regional saturation techniques may also be positioned outside of the central sensor element in any suitable location. FIG. 7 illustrates one such embodiment of a monitoring system 180 having two distinct central emitters 16a disposed in different sensor elements 182b, 182c of an array 181 of sensor elements 182. More specifically, in the depicted embodiment, a sensor element 182a (e.g., a central sensor element) may include a central electrode 20a for BIS monitoring. The sensor element 182a may be removably coupled to a sensor element 182b and/or a sensor element 182c. A first central emitter 16a may be disposed in the sensor element 182b, while a second central emitter 16a may be disposed in the sensor element 182c. Light from the first central emitter 16a may be detected by detectors 18a, 18b disposed in the sensor element 182b, while light from the second central emitter 16a may be detected by detectors 18a, 18b disposed in the sensor element 182c. Thus, two separate sensor elements 182b, 182c may each function as regional saturation sensors and may be configured for regional saturation monitoring. As discussed above with respect to FIG. 5, the sensor elements 182a, 182b, 182c may be removably coupled such as by a perforated edge 154, for example. Thus, one or both of the sensor elements 182b, 182c may be easily separated from the sensor element 182a. In some cases, the sensor elements 182b, 182c may be separated prior to placing the sensor elements 182b, 182c on the patient. However, in some cases, the sensor elements 182a, 182b, 182c may be placed on the patient as a single unit of attached sensor elements 182, and the various portions or sensor elements 182 may be removed for replacement, repair, or to adapt the system 180 to the particular monitoring needs of the patient. Furthermore, the sensor elements 182a, 182b, 182c may optionally share a battery and/or a wireless transceiver, as described above with respect to the sensor elements 152a, 152b, 152c of FIG. 5.

Additionally, the sensor elements 182 may have a shape that improves conformability of the sensor element 182 to the patient's tissue. Such configurations may be particularly useful in configurations having relatively large sensor elements 182, such as the sensor elements 182b, 182c of FIG. 7. For example, as shown in FIG. 7, the sensor elements 182b, 182c have one or more notches that allow the sensor elements 182b, 182c to easily curve or conform to the patient's forehead.

Various methods of applying the sensor elements of the present disclosure are envisioned. In certain systems, the sensor elements may be individually applied to the patient. For example, the operator may first align a central sensor element centrally on the patient's forehead. Then, the operator may align a second sensor element adjacent to the central element, such that the respective components are suitably aligned (e.g., in the case of the system 120 of FIG. 4, the detectors 18a, 18b of the sensor element 122b are aligned with the central emitter 16a of the sensor element 122a along the horizontal axis 130, and the detectors 18a, 18b are about 30 mm and about 40 mm, respectively, from the emitter 16a). Once the central sensor element and the second sensor element are properly aligned, the operator may then apply a third sensor element, and so on. However, in such cases, it may be difficult to attain the proper alignment and distance between the sensor elements and/or the various sensor components within the elements.

Thus, one or more of the sensor elements may be manufactured and/or provided to a healthcare facility in a form that facilitates proper positioning of the sensor elements. For example, in some embodiments, some or all of the sensor elements may be coupled together by perforated edges (as discussed above) with the various components (e.g., emitters, detectors, electrodes) at preferred relative locations. In some embodiments, some or all of the sensor elements may be coupled (e.g., temporarily coupled) together by one or more removable liners (e.g., adhesive sheets, etc.). Such a configuration may be understood with particular attention to FIG. 8, which shows a side cross-sectional view of a portion of a monitoring system 200 having a plurality of sensor elements 202 coupled by two removable liners 204, 206. As shown, three sensor elements 202a, 202b, 202c are disposed at suitable distances and suitable relative positions between a bottom liner 204 and a top liner 206. The sensor element 202a may include two central emitters 16a. As described above with respect to FIG. 4, certain distances between emitters 16a and detectors 18b, 18c may be desirable. Thus, the sensor element 202b may be disposed between the bottom liner 204 and the top liner 206 so that the first detector 18a is a first distance, D₁, from the first central emitter 16a. Additionally, the second detector 18b of the sensor element 202b may be a second distance D₂ from the first central emitter 16a, wherein the distance D₁ is shorter than the distance D₂. In certain embodiments, the first distance D₁ is about 75% of the second distance D₂. In a particular embodiment, the first distance D₁ is about 30 mm while the second distance D₂ is about 40 mm. Thus, the detectors 18a, 18b of the second sensor element 202b may be spaced about 10 mm apart. In other embodiments, the first distance D₁ may be between about 1 cm to about 3 cm, while the second distance D₂ may be about 3 cm to about 4 cm. The sensor element 202c may be similarly constructed and may also be disposed between the bottom liner 204 and the top liner 206 so that the first detector 18a is a first distance, D₁, from the second central emitter 16a and the second detector 18b is a second distance D₂ from the second central emitter 16a.

To apply the sensor elements 202 to the patient, the operator may remove the bottom liner 204, revealing an adhesive bottom surface 208 of each sensor element 202. After removing the bottom liner 204, the sensor elements 202 are still coupled together by the top liner 206, thus the preset, suitable distances and relative positions of the sensor elements 202 are maintained. The operator may apply the sensor elements 202 to the patient, aligning the sensor element 202a centrally on the patient's forehead and pressing the sensor elements 202b, 202c into place, for example. Once the sensor elements 202 are applied to the patient, the operator may remove the top liner 206. This procedure may ensure proper relative positioning of the sensor elements 202 and the components (e.g., emitters 16, detectors 18, etc.) therein, while still providing the benefits of the separate wireless sensor elements 202 after the liners 204, 206 are removed.

Additionally, in some embodiments, removal of the top liner 206 may reveal or provide an adhesive top surface 210 on one or more of the sensor elements 202. Thus, a wrap (e.g., headband) may be applied and adhered to the adhesive top surface 210 of one or more of the sensor elements 202 to protect and/or secure the sensor element 202 to the patient, in certain embodiments. Alternatively, the adhesive top surface 210 may be utilized to attach a wireless transceiver, battery, and/or other components to each sensor element 202, if not otherwise coupled to or included within the sensor element 202. In certain cases, it may be beneficial to provide the wireless transceiver and/or battery as a detachable component that can be removably coupled to each sensor element 202 (as opposed to an integrated component disposed within the sensor element). Such a configuration would allow the wireless transceiver and/or battery to be replaced or repaired more easily, or may allow these more expensive components (e.g., wireless transceivers and batteries) to be reused even if the sensor elements 202 are to be discarded (e.g., disposable sensor elements).

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A multi-component wireless sensor element system comprising:
a medical monitor; and
an array of a plurality of sensor elements, wherein each of the plurality of sensor elements are physically separate from each other and are configured to separately wirelessly communicate with the medical monitor, and two or more of the sensor elements are configured to function together to monitor a physiological parameter.

2. The system of claim 1, wherein at least one of the sensor elements is configured for monitoring at least two physiological parameters.

3. The system of claim 2, wherein the at least one sensor element is configured for bispectral index monitoring and regional saturation monitoring.

4. The system of claim 1, wherein the two or more sensor elements are configured to function together for bispectral index monitoring or regional saturation monitoring.

5. The system of claim 1, wherein at least one of the sensor elements is configured for pulse oximetry monitoring.

6. The system of claim 1, wherein each of the plurality of sensor elements comprises a battery.

7. The system of claim 1, wherein each of the plurality of sensor elements comprises a wireless transceiver.

8. A wireless medical sensor comprising:
a first sensor element comprising an emitter configured to emit light; and
a second sensor element comprising a detector configured to detect the light emitted by the emitter of the first sensor element, wherein the second sensor element is physically separate from the first sensor element;
wherein the first sensor element is coupled to a first wireless transceiver and the second sensor element is coupled to a second wireless transceiver.

9. The sensor of claim 8, wherein each wireless transceiver is configured to separately wirelessly communicate with a medical monitor.

10. The sensor of claim 8, wherein the first sensor element is configured to wirelessly communicate with the second sensor element via the first and second wireless transceivers, and wherein the first sensor element is configured to wirelessly communicate with a medical monitor and to provide physiological data obtained by the first and second sensor elements to the medical monitor via the first wireless transceiver.

11. The sensor of claim 8, wherein the first wireless transceiver is integrated into the first sensor element.

12. The sensor of claim 8, wherein the first wireless transceiver is disposed on a surface of the first sensor element and is electrically connected to the first sensor element.

13. The sensor of claim 8, wherein the first sensor element is coupled to a first battery and the second sensor element is coupled to a second battery.

14. The sensor of claim 8, wherein together the first and second sensor elements are configured for regional saturation monitoring.

15. The sensor of claim 8, wherein the first sensor element comprises an electrode configured for bispectral index monitoring.

16. The sensor of claim 13, comprising a third sensor element comprising electrodes configured for bispectral index monitoring, wherein the third sensor element is physically separate from the first and second sensor elements.

17. A wireless medical sensor comprising:
a first sensor element comprising one or more electrodes; and
a second sensor element comprising one or more electrodes, wherein the second sensor element is physically separate from the first sensor element;
wherein the first sensor element is coupled to a first wireless transceiver and the second sensor element is coupled to second wireless transceiver for wirelessly communicating with a medical monitor, and together the first sensor element and the second sensor element form a sensor configured for wireless bispectral index monitoring.

18. The sensor of claim 17, wherein at least one of the first sensor element or the second sensor element comprises an emitter.

19. The sensor of claim 18, comprising a third sensor element comprising a detector, and together the emitter of the first sensor element or the second sensor element and the detector of the third sensor element are configured for regional saturation monitoring.

20. The sensor of claim 19, comprising a fourth sensor element comprising an emitter and a detector, the fourth sensor element being configured for pulse oximetry monitoring, wherein the fourth sensor element is physically separate from the first, second, and third sensor elements.
